(19) United States
(12) Patent Application Publication
(10) Pub. No.: US 2010/0262145 A1
(43) Pub. Date: Oct. 14, 2010

(54) Medical cutting device and medical cutting training device

(75) Inventors: Ryosuke Kaji, Kyoto-shi (JP); Hirofumi Jikuhara, Kyoto-shi (JP)

Correspondence Address: Quinn Emanuel Urquhart & Sullivan, LLP;Koda/Androlia, 10th Floor, 865 S. Figueroa Street, Los Angeles, CA, 90017, US

(73) Assignee: J. Morita Manufacturing Corporation

(21) Appl. No.: 12/798,889

(22) Filed: Apr. 13, 2010

(30) Foreign Application Priority Data
Apr. 14, 2009 (JP) 2009-097754

(51) Int. Cl. A61B 17/00

(52) U.S. Cl. 606/79

(57) ABSTRACT

Provided is a medical cutting device including a housing, a cutting tool, a tool supporting unit and a load detecting unit. The housing supports the tool supporting unit and the load detecting unit. The tool supporting unit supports the cutting tool. The load detecting unit detects a load applied to the cutting tool.

### BACKGROUND OF THE INVENTION

1. Field of the Invention

The present invention relates to a medical cutting device and a medical cutting training device including the same.

2. Description of the Background Art

In dental treatments, a treatment to cut teeth with a handpiece which is a medical cutting device is performed. It is required to complete a cutting training for acquiring this medical technique. In the cutting training, as described in Japanese Patent Application Laid-Open No. 10-97187, a shape of an artificial tooth which is cut after training is measured, and the measured shape is compared with a cutting shape of a trainer, to thereby make evaluations.

Further, Japanese Patent Application Laid-Open No. 2000-298427 proposes the method of recording a trail of a handpiece with an imaging camera for making real-time evaluations of cutting training.

### SUMMARY OF THE INVENTION

In the cutting training described in Japanese Patent Application Laid-Open No. 10-97187, evaluations are made only after the shape of the artificial tooth which is cut after training is measured, and thus real-time evaluations of cutting cannot be made. In addition, the cutting training described in Japanese Patent Application Laid-Open No. 10-97187 requires an expensive device such as a three-dimensional shape measuring device for measuring a shape of an artificial tooth.

Further, in the cutting training described in Japanese Patent Application Laid-Open No. 2000-298427, it is required to provide a target in a handpiece for recording a trail of the handpiece with ease. In addition, in the cutting training described in Japanese Patent Application Laid-Open No. 2000-298427, a plurality of imaging cameras need to be provided for recording a trail of the handpiece. Moreover, in the cutting training described in Japanese Patent Application Laid-Open No. 2000-298427, a trail of the handpiece is recorded, but a load applied to a cutting tool during cutting cannot be measured.

In a clinical practice, it is possible to avoid excessive cutting if a load applied to a cutting tool during cutting is obtained. However, workability is valued in a clinical practice, and thus it is required to provide a handpiece with a configuration for measuring a load applied to a cutting tool during cutting in a handpiece within a range of, for example, the same shape as that of a normally-used handpiece.

An object of the present invention is therefore to provide a medical cutting device capable of instantly achieving a load applied to a cutting tool during cutting without affecting workability, and a medical cutting training device for making evaluations on training based on the load applied to the cutting tool during cutting.

In order to solve the above-mentioned problems, a medical cutting device according to a first aspect of the present invention includes: a cutting tool; a tool supporting unit supporting the cutting tool; a load detecting unit detecting a load applied to the cutting tool; and a housing supporting the tool supporting unit and the load detecting unit.

According to a second aspect of the present invention, in the medical cutting device according to the first aspect, the load detecting unit includes a bending member having one end fixed to the housing and another end fixed to one of the tool supporting unit and the cutting tool, and a strain detecting unit provided on the bending member and detecting a strain of the bending member; and the load detecting unit detects the load applied to the cutting tool based on the strain of the bending member which is detected by the strain detecting unit.

According to a third aspect of the present invention, in the medical cutting device according to the second aspect, the bending member is fixed to the housing and at least one of the tool supporting unit and the cutting tool via an indirect member.

According to a fourth aspect of the present invention, in the medical cutting device according to the second aspect, the bending member includes a plurality of bending members each including the strain detecting unit; and the respective bending members are disposed by being displaced a predetermined angle with respect to each other, with a long axis of the cutting tool being a center.

According to a fifth aspect of the present invention, in the medical cutting device according to the first aspect, the load detecting unit is sandwiched between the housing and one of the tool supporting unit and the cutting tool; and the load detecting unit detects the load applied to the cutting tool based on a pressure detected by the pressure detecting unit.

According to a sixth aspect of the present invention, in the medical cutting device according to the fifth aspect, the pressure detecting unit is sandwiched between the housing and at least one of the tool supporting unit and the cutting tool via an indirect member.

According to a seventh aspect of the present invention, in the medical cutting device according to the fifth aspect, the load detecting unit includes a plurality of load detecting units; and the pressure detecting units are disposed in a long axis direction of the cutting tool and a direction perpendicular thereto.

According to an eighth aspect of the present invention, in the medical cutting device according to any one of the first to seventh aspects, the housing includes a holding unit and a head unit provided at a tip of the holding unit; the tool supporting unit is provided inside the head unit so that a rotation axis of the cutting tool is provided with a predetermined angle with respect to a long axis of the holding unit; and the head unit includes an air turbine driving unit therein.

According to a ninth aspect of the present invention, in the medical cutting device according to any one of the first to seventh aspects, the housing includes a holding unit and a head unit provided at a tip of the holding unit; the tool supporting unit is provided inside the head unit so that a rotation axis of the cutting tool is provided with a predetermined angle with respect to a long axis of the holding unit; and the holding unit includes a micromotor therein.

According to a tenth aspect of the present invention, in the medical cutting device according to any one of the first to seventh aspects, the cutting tool is a scaler chip.

According to an eleventh aspect of the present invention, in the medical cutting device according to any one of the first to tenth aspects, the medical cutting device further includes a display unit displaying one of the load and a load vector detected by the load detecting unit.

According to a twelfth aspect of the present invention, in the medical cutting device according to the eleventh aspect, the display unit has a function of displaying one of the loads and the load vectors detected by the load detecting unit in chronological order.

According to a thirteenth aspect of the present invention, in the medical cutting device according to any one of the first to tenth aspects, the medical cutting device further includes a notifying unit notifying a fact that the load detected by the load detecting unit is equal to or larger than a predetermined value.

A medical cutting training device according to a fourteenth aspect of the present invention includes: the medical cutting device according to any one of the first to thirteenth aspects; a judging unit receiving one of the load and the load vector detected by the load detecting unit from the medical cutting device, and comparing one of the load and the load vector with a predetermined judgment standard to make evaluations and judgments of a cutting operation; and an output unit outputting evaluation and judgment results by the judging unit.

According to the medical cutting device of the first aspect of the present invention, the load detecting unit capable of detecting a load applied to the cutting tool is provided within the housing, which makes it possible to instantly obtain a load applied to the cutting tool during cutting without affecting workability.

According to the medical cutting device of the second aspect of the present invention, a strain of the bending member is detected by the strain detecting unit, and a load applied to the cutting tool is detected based on the detection result, which enables miniaturization of the load detecting unit.

According to the medical cutting device of the third aspect of the present invention, the bending members are fixed to the housing and the tool supporting unit or the cutting tool via the indirect member, which increases the degree of freedom in layout of components such as the bending member and the tool supporting unit within the medical cutting device.

According to the medical cutting device of the fourth aspect of the present invention, a plurality of bending members each provided with the strain detecting unit are provided in a predetermined arrangement, and hence it is possible to detect loads applied to the cutting tool in various directions.

According to the medical cutting device of the fifth aspect of the present invention, the load applied to the cutting tool is detected based on a pressure detected by the pressure detecting unit, which simplifies the configuration of the load detecting unit.

According to the medical cutting device of the sixth aspect of the present invention, the pressure detecting unit is fixed to the housing and the tool supporting unit or the cutting tool via the indirect member, which increases the degree of freedom in layout of components such as the pressure detecting unit and the tool supporting unit within the medical cutting device.

According to the medical cutting device of the seventh aspect of the present invention, a plurality of pressure detecting units are provided in a predetermined arrangement, which enables detection of loads applied to the cutting tool in various directions.

According to the medical cutting device of the eighth or ninth aspect of the present invention, the cutting tool is capable of being driven by an air turbine or a micromotor.

According to the medical cutting device of the tenth aspect of the present invention, the cutting tool may be a scaler chip.

According to the medical cutting device of the eleventh aspect of the present invention, a load or a load vector is displayed on the display unit, whereby an operator is capable of easily obtaining the load or the load vector applied to the cutting tool during a cutting operation.

According to the medical cutting device of the twelfth aspect of the present invention, the loads or the load vectors are displayed on the display unit in chronological order, whereby the operator is capable of easily obtaining changes with time in load or load vector applied to the cutting tool during a cutting operation.

According to the medical cutting device of the thirteenth aspect of the present invention, the notifying unit notifies the operator of the fact that a load applied to the cutting tool is equal to or larger than a predetermined value, which avoids excessive cutting.

According to the medical cutting device of the fourteenth aspect of the present invention, the medical cutting device capable of detecting a load applied to a cutting tool or a load vector is used, which enables instant evaluations of cutting training based on objective data.

These and other objects, features, aspects and advantages of the present invention will become more apparent from the following detailed description of the present invention when taken in conjunction with the accompanying drawings.

### BRIEF DESCRIPTION OF THE DRAWINGS

FIG. 1 is a cross-sectional view of a medical cutting device according to a first preferred embodiment;

FIG. 2 is a plan view of a load detecting unit of the medical cutting device according to the first preferred embodiment;

FIG. 3 is a view for describing load detection of the medical cutting device according to the first preferred embodiment;

FIG. 4 is another view for describing load detection of the medical cutting device according to the first preferred embodiment;

FIG. 5 is a block diagram of the medical cutting device according to the first preferred embodiment;

FIG. 6 is a schematic cross-sectional view showing a more overall part of the air turbine handpiece;

FIG. 7 is a cross-sectional view of a medical cutting device according to a modification of the first preferred embodiment;

FIG. 8 is a plan view of a load detecting unit of the medical cutting device according to the modification of the first preferred embodiment;

FIG. 9 is another cross-sectional view of the medical cutting device according to the modification of the first preferred embodiment;

FIG. 10 is a schematic cross-sectional view showing a more overall part of the micromotor handpiece;

FIG. 11 is a cross-sectional view of a medical cutting device according to a second preferred embodiment;

FIG. 12 is a cross-sectional view of a load detecting unit of the medical cutting device according to the second preferred embodiment;

FIG. 13 is a cross-sectional view of a medical cutting device according to a modification of the second preferred embodiment; and

FIG. 14 is a block diagram of a medical cutting training device according to a third preferred embodiment.

### DESCRIPTION OF THE PREFERRED EMBODIMENTS

### First Preferred Embodiment

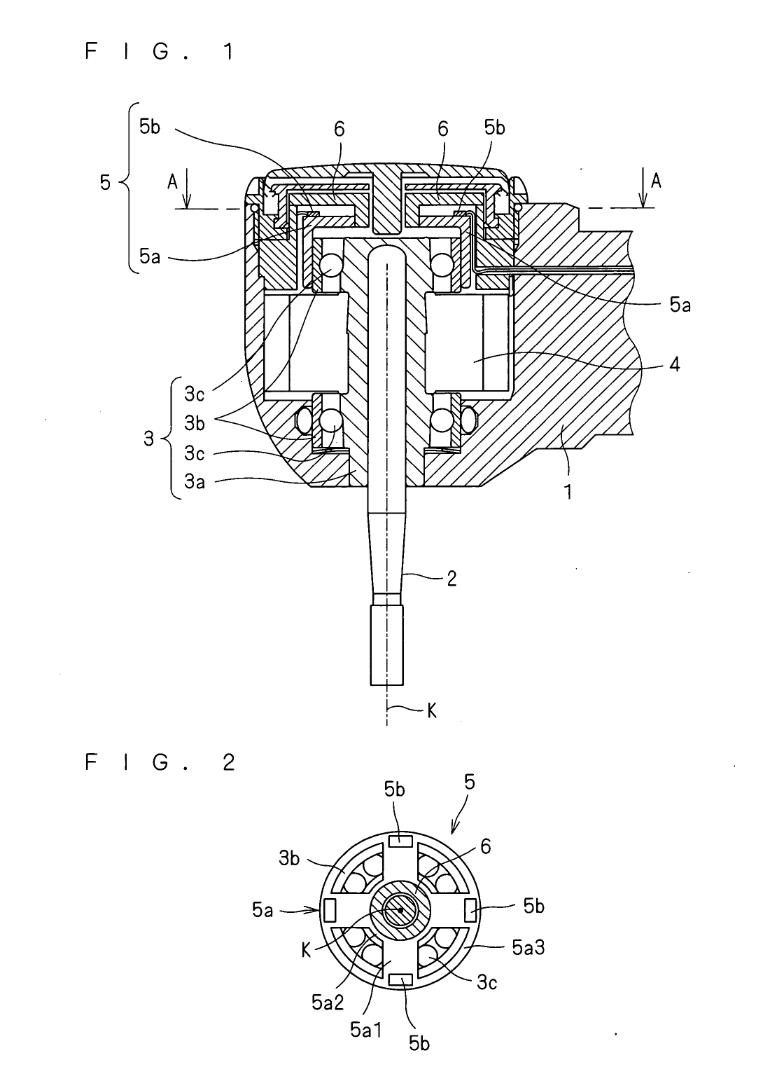

In a medical cutting device according to this preferred embodiment, a load detecting unit which detects a load applied to a cutting tool is provided in a housing. Specific description will be given of a configuration using a strain gauge as a load detecting unit in the medical cutting device according to this preferred embodiment. FIG. 1 is a cross-sectional view of an air turbine handpiece which is the medical cutting device according to this preferred embodiment. The air turbine handpiece shown in FIG. 1 houses, in a housing 1, a cutting tool supporting unit 3 which supports a cutting tool 2, a turbine rotor 4 for driving the cutting tool 2, and a load detecting unit 5 which detects a load applied to the cutting tool 2.

The housing 1 is formed of the same material and has approximately the same shape and scale as those of a housing of a conventionally-used air turbine handpiece. Note that compared with a conventional housing, the housing 1 needs to obtain more space by an amount for housing the load detecting unit 5 therein.

The cutting tool 2 may be any tool as long as it is a cutting tool capable of being attached to and detached from a conventionally-used air turbine handpiece. The cutting tool 2 is defined by JIS T 5501. Stainless steel is used for a shaft part of the cutting tool 2. A diamond wheel, tungsten carbide or the like subjected to nickel plating or chrome plating is used for an operation part of the cutting tool 2. Note that, generally, an operation part of the cutting tool 2 which is composed of diamond wheel, an operation part which is composed of tungsten carbide, an operation part which is obtained by forming stainless steel into a knife shape, and an operation part whose tip is formed of a disc-shaped brush or the like are referred to as “diamond bur”, “carbide bur”, “stainless bur” and “disc”, respectively.

The cutting tool supporting unit 3 includes an inner side supporting unit 3a which directly supports the cutting tool 2, an outer side supporting unit 3b fitted with the housing 1, and bearings 3c sandwiched between the inner side supporting unit 3a and the outer side supporting unit 3b. The inner side supporting unit 3a supports the cutting tool 2 in a detachable manner. In addition, the inner side supporting unit 3a is configured so as to be rotatable with respect to the outer side supporting unit 3b via the bearings 3c. Note that the cutting tool supporting unit 3 shown in FIG. 1 is configured such that two vertical pairs of bearings 3c are used in a long axis K direction of the cutting tool 2 to make the inner side supporting unit 3a rotatable with respect to the outer side supporting part 3b.

The rotor 4 is directly mounted to the inner side supporting unit 3a between the two vertical pairs of bearings 3c as shown in FIG. 1. Accordingly, the rotor 4 is rotated by means of compressed air for driving which is supplied to the air turbine handpiece, whereby the inner side supporting unit 3a is rotated to drive the cutting tool 2.

The load detecting unit 5 includes bending members 5a and strain gauges 5b attached onto the bending members 5a. The bending member 5a has one end fixed to the housing 1 and the other end fixed to the cutting tool supporting unit 3. A load is applied to the cutting tool 2, and a position of the cutting tool supporting unit 3 deviates, whereby strain is caused to the bending member 5a. This strain is detected by the strain gauge 5b, and thus a load applied to the cutting tool 2 is obtained. Note that in the load detecting unit 5, the bending members 5a are fixed to the housing 1 via an indirect member 6. The provision of the indirect member 6 increases the degree of freedom in a position where the bending member 5a is provided, which makes it easier to lay out the load detecting unit 5 in the housing 1. FIG. 1 illustrates the example in which the indirect member 6 is provided on the housing 1 side, but the present invention is not limited thereto. The indirect member 6 may be provided on the cutting tool supporting unit 3 side, or on both the housing 1 side and the cutting tool supporting unit 3 side. In addition, the bending members 5a are fixed to the outer side supporting unit 3b in the load detecting unit 5 shown in FIG. 1, but the present invention is not limited thereto. The bending members 5a may be fixed so as to be rotatable with the inner side supporting unit 3a or the cutting tool 2.

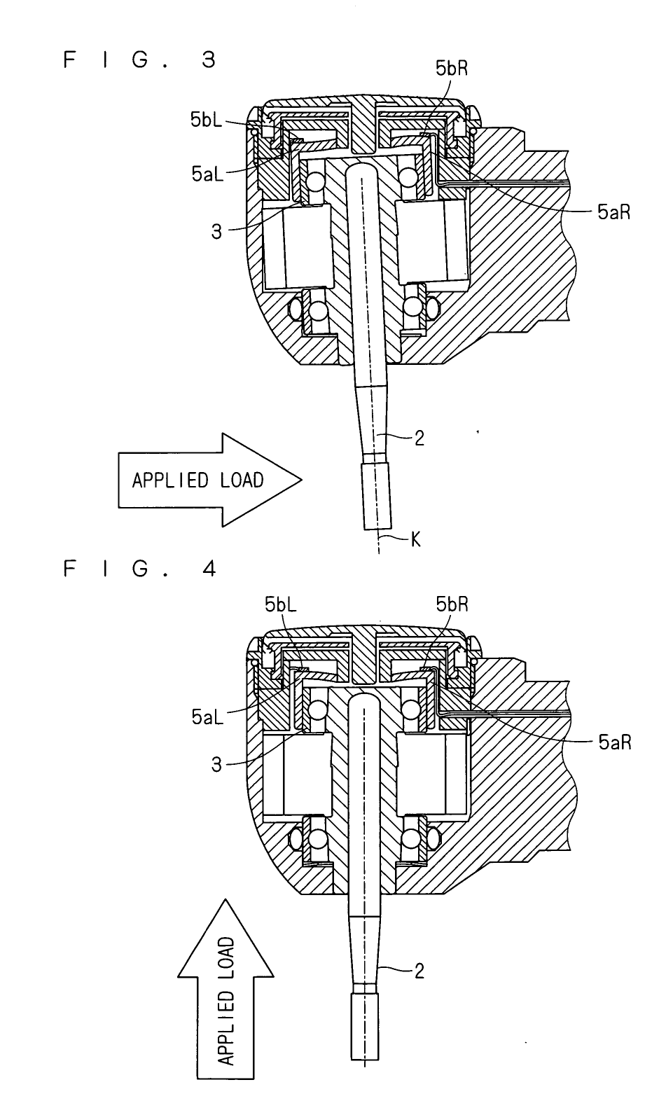

In order to describe the load detecting unit 5 more specifically, FIG. 2 is a plan view of the load detecting unit 5 which is viewed from the A-A plane of FIG. 1. In the load detecting unit 5 shown in FIG. 2, the bending member 5a includes a cross-shaped part 5a1, and the center part of this cross-shaped part 5a1 is fixed to the housing 1 via the indirect member 6. In addition, in the load detecting unit 5 shown in FIG. 2, a periphery part of the cross-shaped part 5a1 is fixed to the outer side supporting unit 3b. Further, in the load detecting unit 5 shown in FIG. 2, the strain gauges 5b are attached to respective sides of the cross-shaped parts 5a1.

Note that the bending members 5a shown in FIG. 2 have a shape of being connected to each other in such a manner that the center parts of the cross-shaped parts 5a1 thereof are connected by a coupling unit 5a2 and the periphery parts thereof are connected by a coupling unit 5a3. This is the shape of the bending member 5a in view of the efficiency in assembling operation, and four independent bending members 5a may be disposed in a cross shape only in view of the detection of a load applied to the cutting tool 2. In addition, in the load detecting unit 5 shown in FIG. 2, the bending members 5a to which the strain gauges 5a are attached are disposed by being displaced 90° with respect to each other around the long axis K of the cutting tool 2. This is the configuration for allowing loads applied to the cutting tool 2 to be detected in the long axis K direction and a direction perpendicular thereto. However, the bending member 5a to which a given strain gauge 5b is attached and the bending member 5a to which an adjacent strain gauge 5b is attached are not necessarily required to be disposed by being displaced 90° with respect to each other. If the bending members 5a to which the strain gauges 5b are attached are disposed by being displaced a predetermined angle (angle of zero degree or larger) with respect to each other, the load detecting unit 5 is capable of obtaining loads in the long axis K direction and the direction perpendicular thereto by computation in consideration of the angle. The number of the bending members 5a to which the strain gauges 5b are attached is not limited to four.

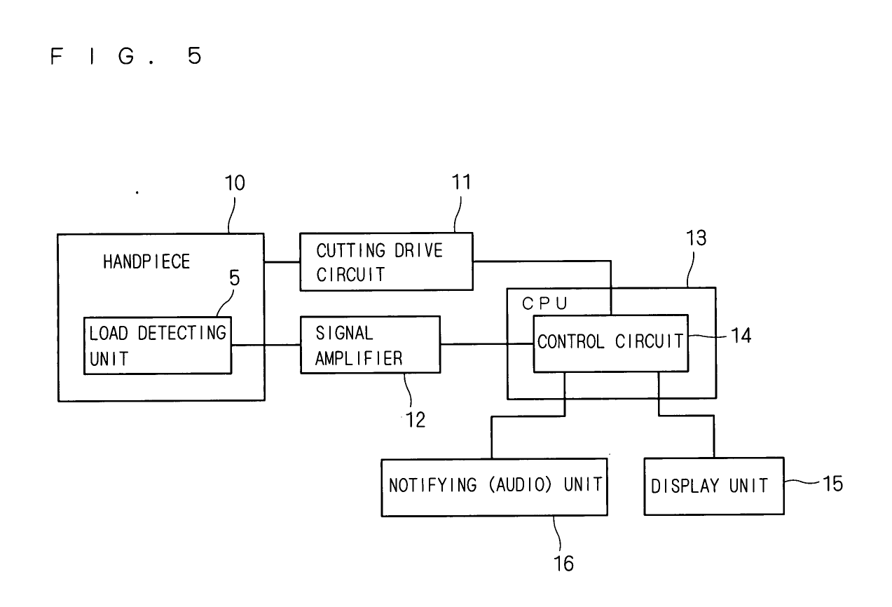

Next, an operation of the load detecting unit 5 will be described. FIG. 3 is a cross-sectional view of an air turbine handpiece in a case where a load is applied to the cutting tool 2 from the left side of the view (in the direction perpendicular to the long axis K). FIG. 4 is a cross-sectional view of the air turbine handpiece in a case where a load is applied to the cutting tool 2 from the lower side of the view (in the long axis K direction).

First, in the air turbine handpiece shown in FIG. 3, the cutting tool 2 is pressed from left to right, and thus the cutting tool supporting unit 3 is tilted leftward. When the cutting tool supporting unit 3 is tilted leftward, strains are caused in a bending member 5aL on the left side of the view and a bending member 5aR on the right side of the view. Accordingly, the load detecting unit 5 shown in FIG. 3 measures the strains caused in the bending members 5aL and 5aR with strain gauges 5bL and 5bR attached thereto, respectively, to thereby obtain a load vector including not only a magnitude of the load applied to the cutting tool 2 but also the direction in which the load is applied.

Next, in the air turbine handpiece shown in FIG. 4, the cutting tool 2 is pressed from down to up, and thus the cutting tool supporting unit 3 is lifted upward. When the cutting tool supporting unit 3 is lifted, strains are caused in the bending member 5aL on the left side of the view and the bending member 5aR on the right side of the view. Accordingly, the load detecting unit 5 shown in FIG. 4 measures the strains caused in the bending members 5aL and 5aR with the strain gauges 5bL and 5bR attached thereto, respectively, to thereby obtain not only the load applied to the cutting tool 2 in the long axis K direction but also the load in the direction perpendicular to the long axis K direction. Note that strains are caused in the bending members 5aL and 5aR also in a case where the cutting tool 2 is pulled from up to down. Note that Japanese Patent Application Laid-Open No. 2004-239621 describes a more specific operation of a load detecting unit (sensor substrate 2 and strain gauge 5).

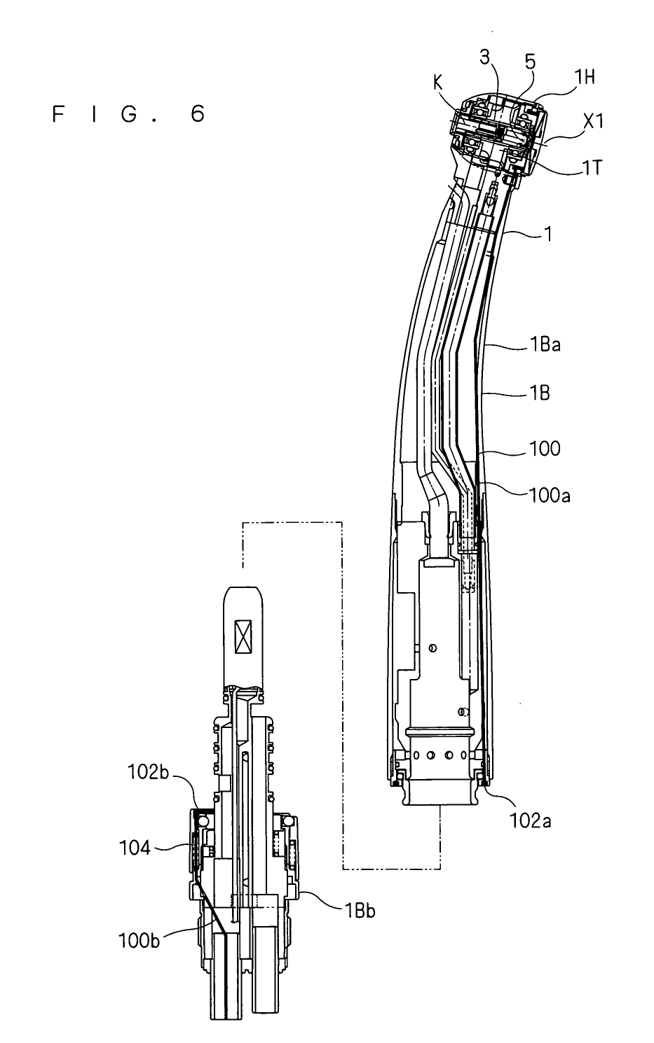

A signal detected by the strain gauge 5b is subjected to a predetermined processing, which is calculated as a load or a load vector applied to the cutting tool 2. The calculated load or load vector is displayed on a display unit. This processing will be described with reference to a block diagram of the medical cutting device according to this preferred embodiment, which is shown in FIG. 5. In the medical cutting device shown in FIG. 5, a cutting drive circuit 11 and a signal amplifier 12 are connected to the handpiece 10 including the load detecting unit 5. The cutting drive circuit 11 and the signal amplifier 12 are connected to a control circuit 14 provided in a CPU 13. The cutting drive circuit 11 controls driving of the cutting tool 2 based on a signal from the control circuit 14. Note that in a case where the handpiece 10 is an air turbine handpiece, in addition to a signal from the cutting drive circuit 11, compressed air for driving is supplied to the handpiece 10.

The signal amplifier 12 is connected to the load detecting unit 5 within the handpiece 10. The signal amplifier 12 amplifies the signal on the strain which has been detected by the load detecting unit 5 to a level capable of being processed by the control circuit 14. Based on the signal amplified by the signal amplifier 12, the control circuit 14 obtains the load or load vector applied to the cutting tool 2 from a strain amount detected by the load detecting unit 5. Note that the load or load vector applied to the cutting tool 2 is preferably obtained in consideration of a length, diameter and material of the cutting tool 2 in the case of being detected from the strain amount detected by the load detecting unit 5.

A total length of the cutting tool 2 is 16 to 70 mm, and a standard length of the standard cutting tool 2 is 19 mm in the air turbine handpiece. Note that normally, the length of the cutting tool 2 is 22 mm in a case of a contra-type micromotor handpiece, and is 44.5 mm in a case of a straight-type cutting tool 2. The diameter of the cutting tool 2 is 1.07 mm to 3 mm, and a standard diameter of the cutting tool 2 is 2.35 mm and 1.6 mm in an air turbine handpiece. In a micromotor handpiece, the diameter of the cutting tool 2 is normally 2.35 mm both in contra type and straight type. As to materials for the cutting tool 2, the axis part thereof is formed of stainless steel, and the operation part thereof is formed of diamond wheel, tungsten carbide or the like subjected to nickel plating or chrome plating.

The load or load vector obtained by the control circuit 14 is displayed on the display unit 15. Note that on the display unit 15, it is possible to display the load and the load vector together with loads and load vectors which have been recorded in a storing unit (not shown) provided in the CPU 13 in chronological order. Further, a value of a load or a load vector which might cause a problem such as excessive cutting is set as a threshold value in the CPU 13, with the result that the medical cutting device according to this preferred embodiment is capable of notifying an operator of the fact that the threshold value is exceeded during cutting by a notifying unit 16 when it occurs. As a result, the operator is capable of obtaining the fact that an excessive load is applied to the cutting tool 2 during cutting. The notifying unit 16 may be configured to make an audio notification or an image notification with the display unit 15.

Further, in the block diagram shown in FIG. 5, the cutting drive circuit 11 and the signal amplifier 12 are provided outside the handpiece 10, but the present invention is not limited thereto. The cutting drive circuit 11 and the signal amplifier 12 may be provided inside the handpiece 10. Alternatively, it is possible to provide the display unit 15 inside the handpiece 10, whereby the operator is capable of obtaining a load applied to the cutting tool 2 without taking his/her eyes off the handpiece at hand.

As described above, the load detecting unit 5 is provided inside the handpiece 10, and thus the medical cutting device according to this preferred embodiment is capable of instantly achieving a load applied to the cutting tool 2 during cutting while suppressing workability from being affected.

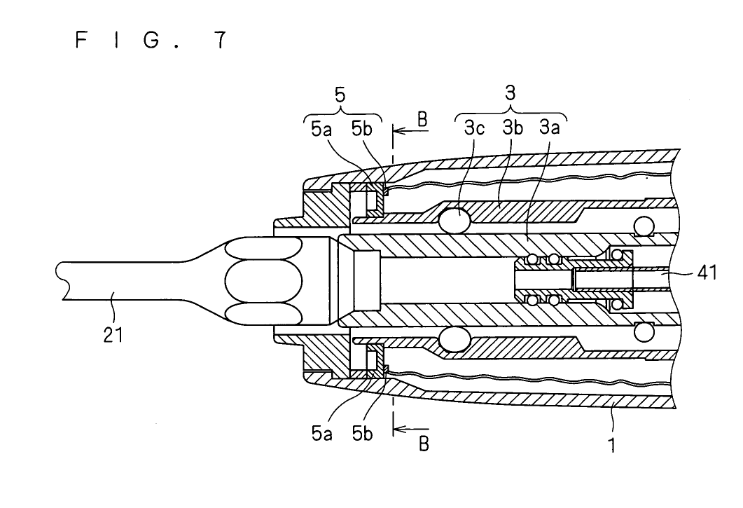

FIG. 6 is a schematic cross-sectional view showing a more overall part of the air turbine handpiece. The housing 1 of the air turbine handpiece includes a holding unit 1B and a head unit 1H. The holding unit 1B is a part capable of being held by a hand. Here, the holding part 1B is formed in a long rod shape, and more specifically, in a long rod shape in such a manner that a part on a tip side thereof is gradually curved. In addition, here, the holding unit 1B is configured to be divided into two parts, a tip-side part 1Ba and a proximal-end-side part 1Bb.

The head unit 1H is provided at a tip part of the holding unit 1B. The cutting tool supporting unit 3 is provided in the head unit 1H. The cutting tool 2 (not shown in FIG. 6, and see FIG. 1 and the like) is supported by the tool supporting unit 3 such that a rotation axis (K) of the cutting tool 2 forms a predetermined angle with respect to a long axis of the holding unit 1B (more specifically, long axis near a part of the holding unit 1B, which is connected to the head part 1H). In other words, the cutting tool 2 supported by the tool supporting unit 3 is oblique with respect to the long axis of the holding unit 1B.

In addition, an air turbine driving unit 1T is provided inside the head unit 1H. The air turbine driving unit 1T is supplied with compressed air through a dedicated tube and the holding unit 1B from a unit main body (not shown). The compressed air causes an impeller of the air turbine driving unit 1T to rotate at high speed, and the rotation is transmitted to the cutting tool 2 via the tool supporting unit 3, whereby the cutting tool 2 rotates at high speed.

The load detecting unit 5 is provided at an upper end of a part of the head unit 1H, which chucks the cutting tool 2. A specific configuration of the load detecting unit 5 itself has already been described above. The air turbine handpiece is provided therein with an electric wire 100 for transmitting a detection signal of the load detecting unit 5 to the unit main body which is externally provided. The electric wire 100 is arranged so as to pass through the holding unit 1B of the housing. For example, in a case where there is provided an interconnection including an electric wire for causing an illumination light provided in the head unit 1H to turn on in the air turbine handpiece, such electric wire 100 may be configured so as to use a part of the interconnection in common.

In the present embodiment, the head unit 1B is configured to be divided into two parts, the tip-side part 1Ba and the proximal-end-side part 1Bb. Accordingly, at a connection part thereof, an electric wire 100a of the tip-side part 1Ba and an electric wire 100b of the proximal-end-side part 1Bb are connected to each other through a contact structure in which the electric wires 100a and 100b are brought into contact with and separated from each other by connection and separation of the tip-side part 1Ba and the proximal-end-side part 1Bb. As the contact structure as described above, there may be employed, for example, a structure in which a ring-shaped contact member 102a is provided on a connection surface of the tip-side part 1Ba and a contact member 102b is disposed on a connection surface of the proximal-end-side part 1Bb to unite and connect the tip-side part 1Ba and the proximal-end-side part 1Bb, which brings the ring-shaped contact member 102a and the contact member 102b into conduction. In this case, at least one of the contact members is desirably biased against the other thereof by a biasing member 104 such as a coil spring. In this case, the contact member 102b is biased by the biasing member 104 in a direction in which connection with the ring-shaped contact member 102a is kept (pressing direction) such that the contact member 102a and the contact member 102b keep a state in which they are always in contact with each other with more reliability. As the biasing member as described above, for example, a ball joint member or the like is used.

(Modification)

The example of the air turbine handpiece has been described in the medical cutting device shown in FIG. 1, but the present invention is not limited thereto. For example, a scaler shown in FIG. 7 is conceivable. Also in the scaler shown in FIG. 7, the housing 1 houses the cutting tool supporting unit 3 which supports a scaler chip 21 serving as a cutting tool, a drive unit 41 for driving the scaler chip 21, and the load detecting unit 5 which detects a load applied to the scaler chip 21.

As in the case of FIG. 1, the cutting tool supporting unit 3 includes the inner side supporting unit 3a which directly supports the scaler chip 21, the outer side supporting unit 3b fitted with the housing 1, and the bearings 3c sandwiched between the inner side supporting unit 3a and the outer side supporting unit 3b.

As in the case of FIG. 1, the load detecting unit 5 includes the bending members 5a and the strain gauges 5b attached onto the bending members 5a. The bending member 5a has one end fixed to the housing 1 and the other end fixed to the outer side supporting unit 3b of the cutting tool supporting unit 3. Note that differently from FIG. 1, the bending members 5a are directly fixed to the housing 1 in the load detecting unit 5 shown in FIG. 7.

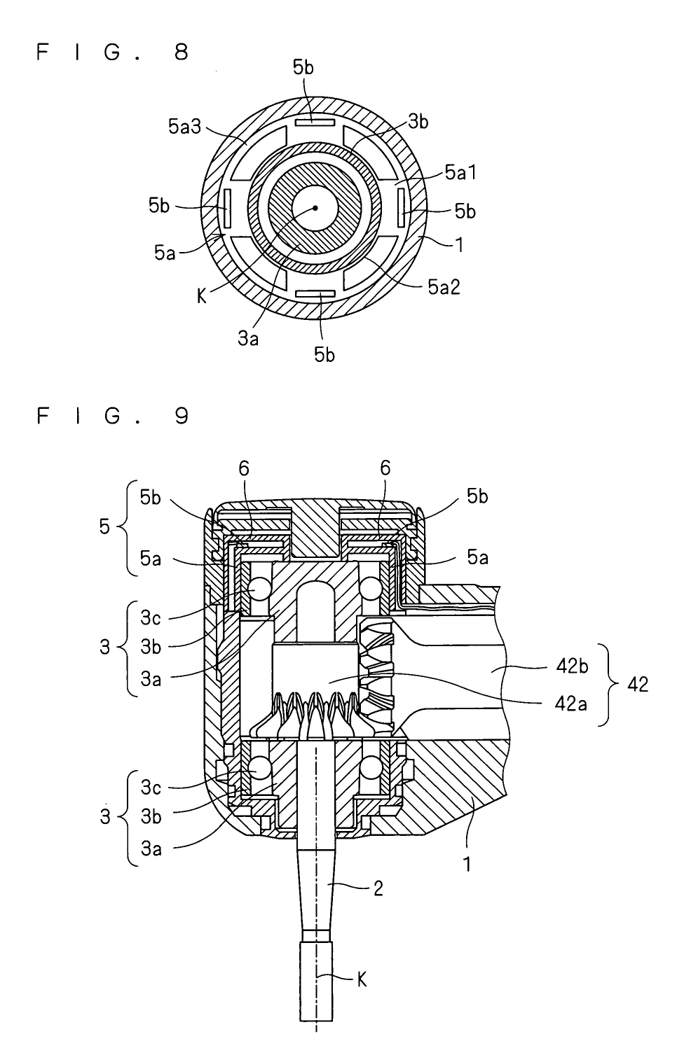

FIG. 8 is a plan view of the load detecting unit 5 which is viewed from the B-B plane of FIG. 7. In the load detecting unit 5 shown in FIG. 8, the bending member 5a includes the cross-shaped part 5a1, and the center part of this cross-shaped part 5a1 is fixed to the outer side supporting part 3b. Further, in the load detecting unit 5 shown in FIG. 8, the periphery part of the cross-shaped part 5a1 is fixed to the housing 1. In addition, in the load detecting unit 5 shown in FIG. 8, the strain gauges 5b are attached to respective sides of the cross-shaped parts 5a1. Note that the bending member 5a shown in FIG. 8 also has the shape such that the center parts of the cross-shaped parts 5a1 are connected to each other with the coupling unit 5a2, and the periphery parts thereof are connected to each other with the coupling part 5a3.

Further, load detection of the scaler shown in FIG. 7 and a signal processing thereof are the same as those of the air turbine handpiece shown in FIG. 1, and thus their detailed descriptions will be omitted. Moreover, the components of the scaler shown in FIG. 7 which are the same as those of the air turbine handpiece shown in FIG. 1 will be denoted by the same configuration numbers, and their detailed descriptions will be omitted.

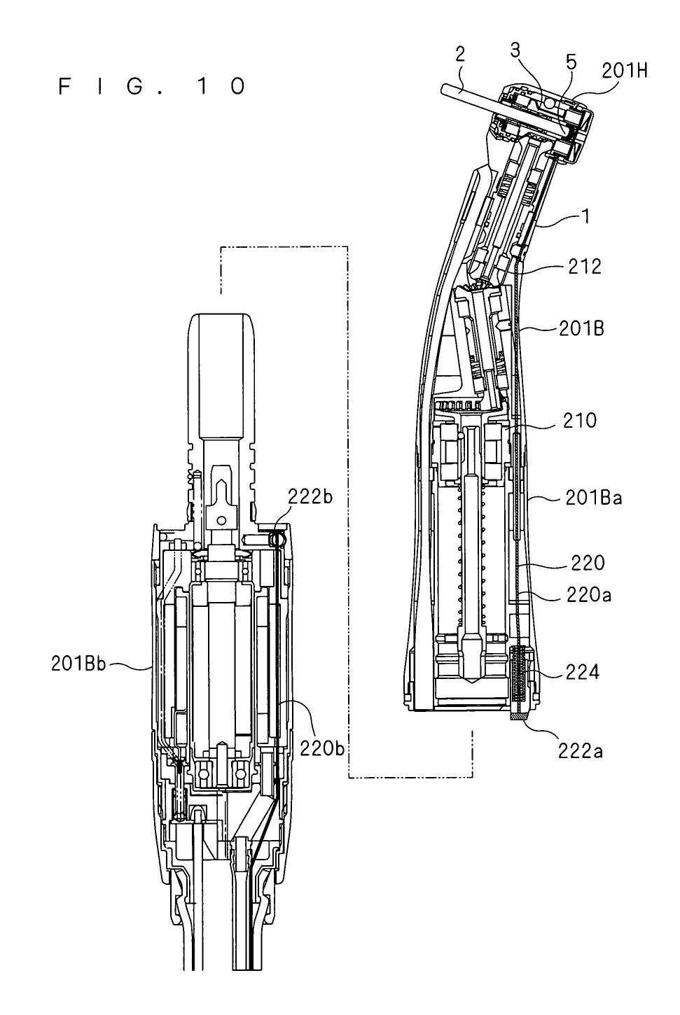

Further, in addition to the air turbine handpiece, a micromotor handpiece shown in FIG. 9 is conceivable. Also in the micromotor handpiece shown in FIG. 9, the housing 1 houses the cutting tool supporting unit 3 which supports the cutting tool 2, a drive mechanism 42 for driving the cutting tool 2, and the load detecting unit 5 which detects a load applied to the cutting tool 2.

As in the case of FIG. 1, the cutting tool supporting unit 3 includes the inner side supporting unit 3a which supports the cutting tool 2, the outer side supporting unit 3b fitted with the housing 1, and the bearings 3c sandwiched between the inner side supporting unit 3a and the outer side supporting unit 3b.

The drive mechanism 42 includes a gear 42a directly mounted onto the inner side supporting unit 3a between two vertical pairs of bearings 3c and a gear 42b meshing with the gear 42a as shown in FIG. 9. The gear 42b is driven by a micromotor (not shown). The micromotor rotates the inner side supporting unit 3a with the drive mechanism 42 to drive the cutting tool 2.

As in the case of FIG. 1, the load detecting unit 5 includes the bending members 5a and the strain gauges 5b attached onto the bending members 5a. The bending member 5a has one end fixed to the housing 1 and the other end fixed to the outer side supporting unit 3b of the cutting tool supporting unit 3. Note that as in the case of FIG. 1, the bending members 5a are fixed to the housing 1 via the indirect member 6 in the load detecting unit 5 shown in FIG. 9.

Further, the shape of the load detecting unit 5, load detection and the signal processing thereof in the micromotor handpiece shown in FIG. 9 are the same as those of the air turbine handpiece shown in FIG. 1, and thus detailed descriptions thereof will be omitted. In addition, the same components of the micromotor handpiece shown in FIG. 9 as those of the air turbine handpiece shown in FIG. 1 are denoted by the same configuration numbers, and their detailed descriptions will be omitted.

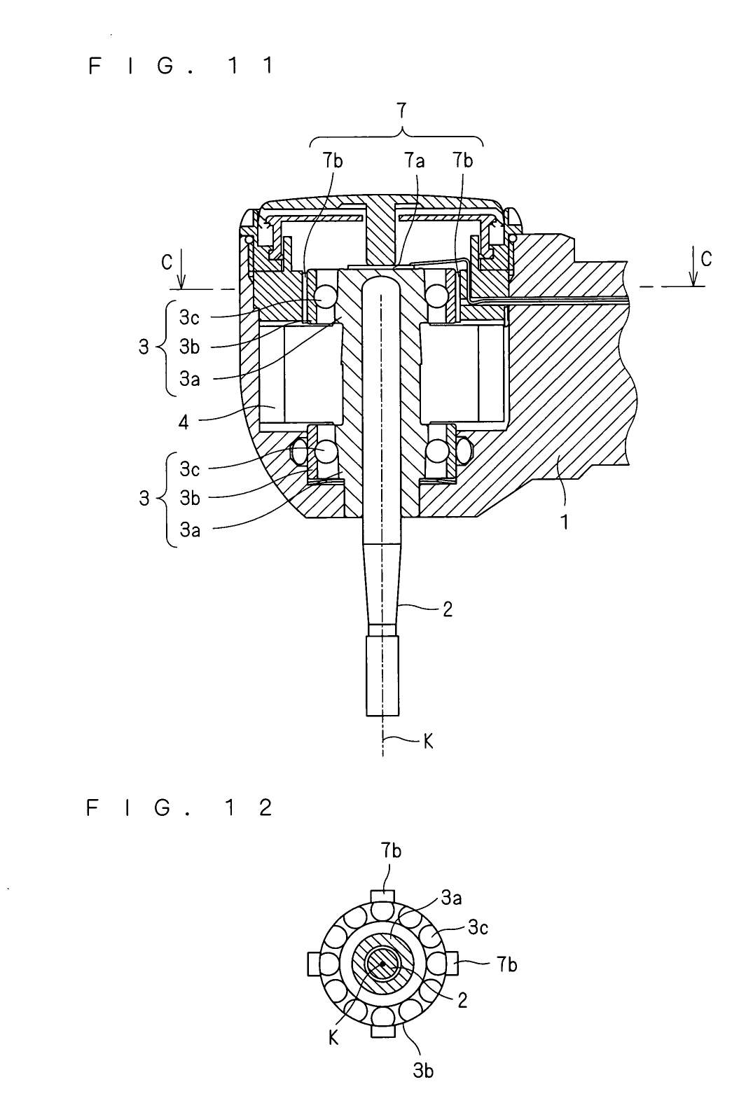

FIG. 10 is a schematic cross-sectional view showing a more overall part of the micromotor turbine handpiece. Here, FIG. 10 shows a contra-angle handpiece.

The housing 1 of the micromotor handpiece includes a holding unit 201B and a head unit 201H. The supporting unit 201B is a part capable of being held by a hand. Here, the holding part 201B is formed in a long rod shape, and more specifically, in a long rod shape in such a manner that a part on a tip side thereof is gradually curved. In addition, here, the holding unit 201B is configured to be divided into two parts, a tip-side part 201Ba and a proximal-end-side part 201Bb.

The head unit 201H is provided at a tip part of the holding unit 201B. The cutting tool supporting unit 3 is provided in the head unit 201H. The cutting tool 2 is supported by the tool supporting unit 3 such that a rotation axis of the cutting tool 2 to be supported forms a predetermined angle with respect to a long axis of the holding unit 201B (more specifically, long axis near a part of the holding unit 1B, which is connected to the head part 201H). In other words, the cutting tool 2 supported by the tool supporting unit 3 is oblique with respect to the long axis of the holding unit 201B. In addition, a micromotor 210 is provided inside the holding unit 201B. A rotation transmission mechanism 212 is provided in a part ranging from the holding unit 201B to the tool supporting unit 3 in the head unit 201H. The rotation transmission mechanism 212 is configured by combining a plurality of gears. The rotation of the micromotor 210 is transmitted to the tool supporting unit 3 via the rotation transmission mechanism 212, whereby the cutting tool 2 is rotated and driven. Note that it is possible to, for example, reduce, keep, or increase speed of the cutting tool 2 through a change in gear ratio of the rotation transmission mechanism 212, at a desired rotational speed.

The load detecting unit 5 is provided at an upper end of a part, which chucks the cutting tool 2, of the head unit 201H. A specific configuration of the load detecting unit 5 itself has already been described above. The micromotor handpiece is provided therein with an electric wire 220 for transmitting a detection signal of the load detecting unit 5 to the unit main body externally provided. The electric wire 220 is arranged so as to pass through the holding unit 201B of the housing. For example, in a case where there is provided an interconnection including an electric wire for driving the micromotor 210 in the micromotor handpiece, such electric wire 220 may be configured so as to use a part of the interconnection in common.

In the present embodiment, the head unit 201B is configured to be divided into two parts, the tip-side part 201Ba and the proximal-end-side part 201Bb. Accordingly, at a connection part thereof, an electric wire 220a of the tip-side part 201Ba and an electric wire 220b of the proximal-end-side part 201Bb are connected to each other through a contact structure in which the electric wires 220a and 220b are brought into contact with and separated from each other by connection and separation of the tip-side part 1Ba and the proximal-end-side part 1Bb. As the contact structure as described above, there may be employed, for example, a structure in which a ring-shaped contact member 222a is provided on a connection surface of the tip-side part 201Ba and a contact member 222b is disposed on a connection surface of the proximal-end-side part 201Bb to unite and connect the tip-side part 201Ba and the proximal-end-side part 201Bb, which brings the contact member 222a and the contact member 222b into conduction. In this case, at least one of the contact members is desirably biased against the other thereof by a biasing member 224 such as a coil spring. In this case, the contact member 222a is biased in a direction in which connection with the contact member 222b is kept (pressing direction) so that the contact member 222b keep a state in which they are always in contact with each other with more reliability.

### Second Preferred Embodiment

Also in a medical cutting device according to this preferred embodiment, as in the first preferred embodiment, the load detecting unit which detects a load applied to the cutting tool is provided inside the housing. Differently from the first preferred embodiment, the medical cutting device according to this preferred embodiment has a configuration in which not the strain gauges, but pressure sensors are used in the load detecting unit. FIG. 11 is a cross-sectional view of an air turbine handpiece which is the medical cutting device according to this preferred embodiment. In the air turbine handpiece shown in FIG. 11, the housing 1 houses the cutting tool supporting unit 3 which supports the cutting tool 2, the turbine rotor 4 for driving the cutting tool 2, and a load detecting unit 7 which detects a load applied to the cutting tool 2. Note that the same components of the air turbine handpiece shown in FIG. 11 as those of the air turbine handpiece shown in FIG. 1 are denoted by the same configuration numbers, and their detailed descriptions will be omitted.

A pressure sensor is used for the load detecting unit 7. Examples of the pressure sensor include a conductive pressure sensor having a film shape or a shape of an elastic body such as rubber and a capacitance type pressure. Note that other than those, any pressure sensor is used as the load detecting unit 7 as long as it is capable of being disposed inside the housing 1. The load detecting unit 7 has one end fixed to the housing 1 and the other end fixed to the cutting tool supporting unit 3. A load is applied to the cutting tool 2, and the position of the cutting tool supporting unit 3 is displaced, whereby the load is applied to the load detecting unit 7. This load is detected by the pressure sensor serving as the load detecting unit 7, to thereby obtain a load applied to the cutting tool 2. Note that in FIG. 11, a load detecting unit 7a disposed in the long axis K direction of the cutting tool 2 has one end fixed to the housing 1 and the other end fixed to the inner side supporting unit 3a of the cutting tool supporting unit 3. In addition, a load detecting unit 7b disposed in the direction perpendicular to the long axis K direction of the cutting tool 2 has one end fixed to the housing 1 and the other end fixed to the outer side supporting unit 3b of the cutting tool supporting unit 3. Note that the load detecting unit 7a shown in FIG. 11 is fixed to the inner side supporting unit 3a, but the present invention is not limited thereto. The load detecting unit 7a may be fixed to the outer side supporting unit 3b or fixed so as to be rotatable with the cutting tool 2. The load detecting unit 7b shown in FIG. 11 is fixed to the outer side supporting unit 3b, but the present invention is not limited thereto. The load detecting unit 7b may be fixed so as to be rotatable with the inner side supporting unit 3a or the cutting tool 2.

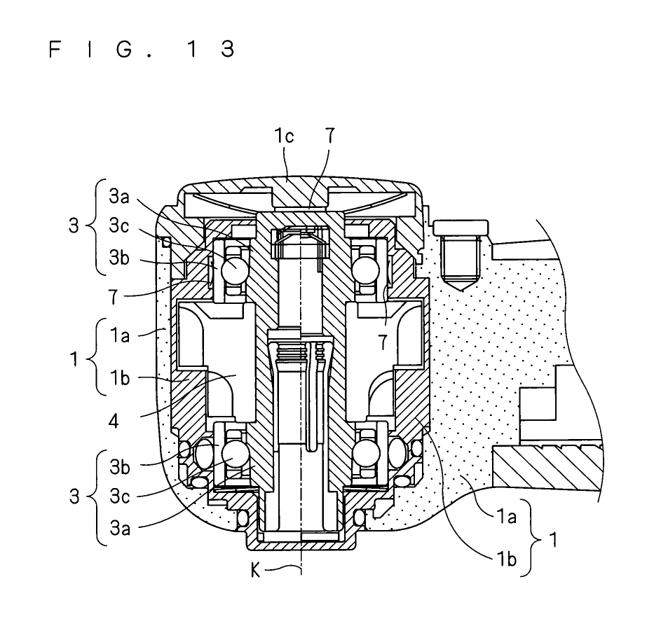

For more detailed description of the load detecting unit 7, FIG. 12 is a cross-sectional view of the load detecting unit 7 in the C-C plane of FIG. 11. The load detecting units 7b shown in FIG. 12 are disposed on the outer side supporting unit 3b by being displaced 90° around the long axis K of the cutting tool 2. Accordingly, the load detecting units 7b shown in FIG. 12 are capable of detecting loads applied to the cutting tool 2 in all directions perpendicular to the long axis K. However, angles between adjacent load detecting units 7b are not necessarily required to be 90°. If the angle between the adjacent load detecting units 7b is determined, a load in the long axis K direction is obtained by computation in consideration of the angle. The number of the load detecting units 7b to be provided is not limited to four. Note that though not shown in FIG. 12, a load in the long axis K direction is detected by the load detecting unit 7a.

Also in the air turbine handpiece shown in FIG. 11, a signal detected by the load detecting unit 7 is processed to obtain a load or a load vector applied to the cutting tool 2 with the configuration shown in FIG. 5. Although it is required to convert a measured strain amount into a load in the air turbine handpiece shown in FIG. 1, the conversion as descried above is not required in the air turbine handpiece shown in FIG. 11 because one to be obtained is an output from the pressure sensor. Note that also in the case of the air turbine handpiece shown in FIG. 11, a load or a load vector applied to the cutting tool 2 is preferably obtained in consideration of a length, diameter, and material of the cutting tool with respect to the load detected by the load detecting unit 7.

As described above, in the medical cutting device according to this preferred embodiment, the load detecting unit 7 formed of a pressure sensor is provided inside the handpiece 10, whereby it is possible to instantly achieve a load applied to the cutting tool 2 during cutting while suppressing workability from being affected.

(Modification)

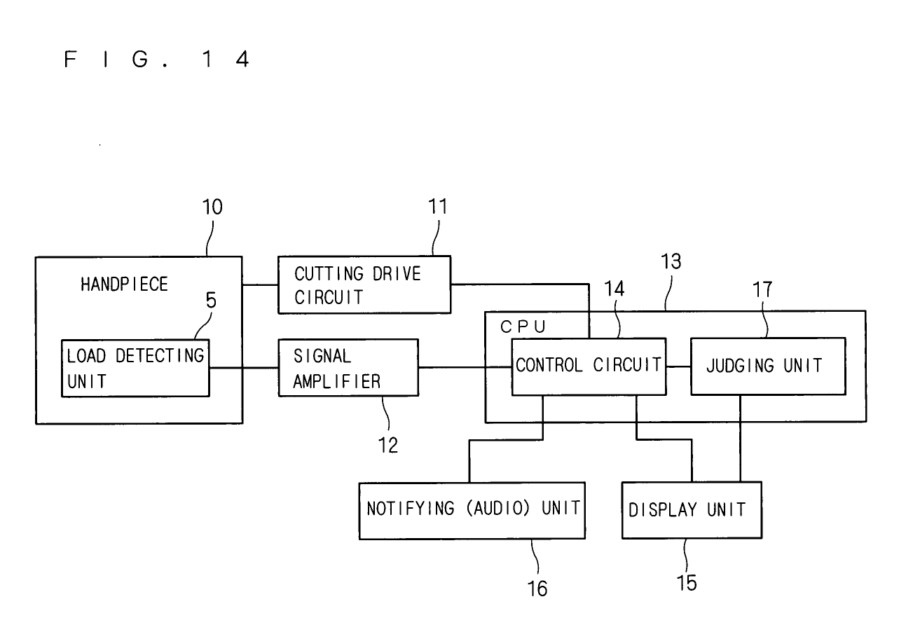

The case where the housing 1 has a single structure has been described in the air turbine handpiece shown in FIG. 11, but the present invention is not limited thereto, and an air turbine handpiece of a double-structure housing 1 as shown in FIG. 13 may be used. The air turbine handpiece shown in FIG. 13 employs the double-structure housing 1 including an outer housing unit 1a positioned on an outer side, an inner housing unit 1b positioned on an inner side, and a capsule cap unit 1c fitted with the outer housing unit 1a. Accordingly, the load detecting unit 7 shown in FIG. 13 has one end fixed to the inner housing unit 1b and the other end fixed to the inner side supporting unit 3a of the cutting tool supporting unit 3. In the air turbine handpiece shown in FIG. 13, the capsule cap unit 1c is capable of being detached in the long axis K direction for taking out the housing unit 1b, which makes maintenance easier. The load detecting unit 7 as shown in FIG. 13 is provided also in the air turbine handpiece in which the double-structure housing 1 is used in this manner, which makes it possible to detect a load or a load vector applied to the cutting tool 2 (not shown in FIG. 13). Note that the same components of the air turbine handpiece shown in FIG. 13 as those of the air turbine handpiece shown in FIG. 11 are denoted by the same configuration numbers, and their detailed descriptions will be omitted. Further, the double-structure housing 1 shown in FIG. 13 is applicable to the air turbine handpiece shown in FIG. 1 which detects a load with the strain gauge 5b in a similar manner.

### Third Preferred Embodiment

The medical cutting device which obtains a load applied to the cutting tool 2 during cutting has been described in the first and second preferred embodiments. The medical cutting device is capable of being used in clinical practice as well as training. Further, description will be given of a medical cutting training device which includes the medical cutting device and is capable of making evaluations of cutting in training.

FIG. 14 is a block diagram of the medical cutting training device according to this preferred embodiment. The medical cutting training device shown in FIG. 14 is configured such that a judging unit 17 is added to the medical cutting device shown in FIG. 5. The judging unit 17 is formed within the CPU 13, and compares a load or a load vector applied to the cutting tool 2 which is obtained from the control circuit 14 with a predetermined judgment standard, to thereby make evaluations and judgments of a cutting operation. As the predetermined judgment standard, there is employed, for example, a standard history of loads or load vectors applied to the cutting tool 2 in cutting of artificial teeth, which has been performed by a trainer. In evaluations and judgments, for example, a training history of loads or load vectors applied to the cutting tool 2 in cutting of artificial teeth, which has been performed by a trainee, with this reference history, to thereby make judgments based on an extent of a statistical difference therebetween. Note that the judging unit 17 may be configured as software in the CPU 13 as shown in FIG. 14, or may be configured as hardware outside the CPU 13.

The evaluation and judgment results are displayed on the display unit 15. Note that other output device such as a printer may be used as an output unit without using the display unit 15 as an output unit of the evaluation and judgment results. In the medical cutting training device shown in FIG. 14, the same components as those of the medical cutting device shown in FIG. 5 are denoted by the same configuration numbers, and their detailed descriptions will be omitted.

As described above, the medical cutting training device according to this preferred embodiment includes the medical cutting device according to the first or second preferred embodiment, the judging unit 17 which makes evaluations and judgments in training with the use of an output of the medical cutting device, and the output unit capable of outputting evaluation results, which enables instant evaluations of cutting training based on objective data.

While the invention has been shown and described in detail, the foregoing description is in all aspects illustrative and not restrictive. It is therefore understood that numerous modifications and variations can be devised without departing from the scope of the invention.

What is claimed is:

1. A medical cutting device, comprising:
a cutting tool;
a tool supporting unit supporting said cutting tool;
a load detecting unit detecting a load applied to said cutting tool; and
a housing supporting said tool supporting unit and said load detecting unit.

2. The medical cutting device according to claim 1, wherein:
said load detecting unit includes:
a bending member having one end fixed to said housing and another end fixed to one of said tool supporting unit and said cutting tool; and
a strain detecting unit provided on said bending member and detecting a strain of said bending member; and
said load detecting unit detects the load applied to said cutting tool based on the strain of said bending member which is detected by said strain detecting unit.

3. The medical cutting device according to claim 2, wherein said bending member is fixed to said housing and at least one of said tool supporting unit and said cutting tool via an indirect member.

4. The medical cutting device according to claim 2, wherein:
said bending member includes a plurality of bending members each including said strain detecting unit; and
the respective bending members are disposed by being displaced a predetermined angle with respect to each other, with a long axis of said cutting tool being a center.

5. The medical cutting device according to claim 1, wherein:
said load detecting unit is sandwiched between said housing and one of said tool supporting unit and said cutting tool; and
said load detecting unit detects the load applied to said cutting tool based on a pressure detected by said pressure detecting unit.

6. The medical cutting device according to claim 5, wherein said pressure detecting unit is sandwiched between said housing and at least one of said tool supporting unit and said cutting tool via an indirect member.

7. The medical cutting device according to claim 5, wherein:
said load detecting unit includes a plurality of load detecting units; and
said pressure detecting units are disposed in a long axis direction of said cutting tool and a direction perpendicular thereto.

8. The medical cutting device according to claim 1, wherein:
said housing includes a holding unit and a head unit provided at a tip of said holding unit;
said tool supporting unit is provided inside said head unit so that a rotation axis of said cutting tool is provided with a predetermined angle with respect to a long axis of said holding unit; and
said head unit includes an air turbine driving unit therein.

9. The medical cutting device according to claim 1, wherein:
said housing includes a holding unit and a head unit provided at a tip of said holding unit;
said tool supporting unit is provided inside said head unit so that a rotation axis of said cutting tool is provided with a predetermined angle with respect to a long axis of said holding unit; and
said holding unit includes a micromotor therein.

10. The medical cutting device according to claim 1, wherein said cutting tool is a scaler chip.

11. The medical cutting device according to claim 1, further comprising a display unit displaying one of the load and a load vector detected by said load detecting unit.

12. The medical cutting device according to claim 11, wherein said display unit displays one of the loads and the load vectors detected by said load detecting unit in chronological order.

13. The medical cutting device according to claim 1, further comprising a notifying unit notifying a fact that the load detected by said load detecting unit is equal to or larger than a predetermined value.

14. A medical cutting training device, comprising:
the medical cutting device according to claim 1;
a judging unit receiving one of the load and the load vector detected by said load detecting unit from said medical cutting device, and comparing one of said load and the load vector with a predetermined judgment standard to make evaluations and judgments of a cutting operation; and
an output unit outputting evaluation and judgment results by said judging unit.
